SEQUENCE LISTING (5 sequences)

SEQ ID NO: 1 - protein, 107 aa, Homo sapiens [sequence omitted]

SEQ ID NO: 2 - protein, 214 aa, Homo sapiens
DIQMTQSPSSLSASVGDRVTITCGTSEDIINYLNWYQQKPGKAPKLLIYHTSRLQSGVPSRFSGSGSGTDFTLTISSLQPEDFATYYCQQGYTLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 121 aa, Homo sapiens [sequence omitted]

SEQ ID NO: 4 - protein, 451 aa, Homo sapiens
EVQLVQSGAEVKKPGASVKVSCKASGYTFTSYVIHWVRQRPGQGLAWMGYINPYNDGTKYNERFKGKVTITSDRSTSTVYMELSSLRSEDTAVYLCGREGIRYYGLLGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 400 aa, Homo sapiens
DLLPDEKISLLPPVNFTIKVTGLAQVLLQWKPNPDQEQRNVNLEYQVKINAPKEDDYETRITESKCVTILHKGFSASVRTILQNDHSLLASSWASAELHAPPGSPGTSIVNLTCTTNTTEDNYSRLRSYQVSLHCTWLVGTDAPEDTQYFLYYRYGSWTEECQEYSKDTLGRNIACWFPRTFILSKGRDWLAVLVNGSSKHSAIRPFDQLFALHAIDQINPPLNVTAEIEGTRLSIQWEKPVSAFPIHCFDYEVKIHNTRNGYLQIEKLMTNAFISIIDDLSKYDVQVRAAVSSMCREAGLWSEWSQPIYVGNDEHKPLREWFVIVIMATICFILLILSLICKICHLWIKLFPPIPAPKSNIKDLFVTTNYEKAGSSETEIEVICYIEKPGVETLEDSVF

(19) United States
(12) Patent Application Publication
(10) Pub. No.: US 2014/0328839 A1
(43) Pub. Date: Nov. 6, 2014

(54) Methods For Reducing The Frequency And Severity Of Acute Exacerbations Of Asthma

(71) Applicant: Medlmmune, LLC, Gaithersburg, MD (US)

(72) Inventors: Nestor Molfino, South San Francisco, CA (US); Joseph Parker, Gaithersburg, MD (US)

(21) Appl. No.: 14/351,796

(22) PCT Filed: Oct. 28, 2012
(86) PCT No.: PCT/US12/62349

§ 371 Date: Apr. 14, 2014

Related U.S. Application Data
(60) Provisional application No. 61/554,110, filed on Nov. 1, 2011.
(60) Provisional application No. 61/562,544, filed on Nov. 22, 2011.
(60) Provisional application No. 61/645,699, filed on May 11, 2012.

(51) Int. Cl. C07K 16/28; A61K 45/06; A61K 39/395

(52) U.S. Cl. CPC C07K 16/2866; A61K 39/3955; A61K 45/06
USPC 424/133.1; 424/172.1; 424/152.1; 424/142.1

(57) ABSTRACT

Provided herein is are methods of reducing the number and severity of acute exacerbations of asthma in an asthma patient, comprising administering to a patient with a history of acute exacerbations of asthma an effective amount of an anti-interleukin-5 receptor (IL-5R) antibody or antigen-binding fragment thereof, for example, an anti-IL-5Rα antibody or antigen-binding fragment thereof, e.g., benralizumab.

### BACKGROUND

More than 300 million people around the world have asthma. Despite the use of long-acting bronchodilators and inhaled corticosteroids, unscheduled visits to doctor offices, visits to emergency departments (ED), and hospitalizations due to asthma exacerbations occur frequently and account for a significant proportion of healthcare costs attributable to asthma. (Masoli M, et al. Allergy 59: 469-78(2004)).

Relapse following acute asthma exacerbation has been reported to range from 41 to 52% at 12 weeks despite the use of systemic steroids upon discharge (Lederle F, et al. Arch Int Med 147:2201-03 (1987)). Management of these patients has proved problematic due either to severe refractory disease or inability and/or unwillingness to comply with medical treatment. In one study of patients admitted to the hospital, some with near fatal asthma, 50% were non-compliant with systemic corticosteroids at 7 days following discharge (Krishnan J, et al. AJRCCM 170: 1281-85 (2004)). Many factors may contribute to non-compliance including poor access to routine quality healthcare (particularly in the inner city), lack of education or understanding of their disease, unwillingness to accept the chronic nature of their disease, or inability to obtain medications.

Many lines of evidence implicate eosinophils as one of the main causative cells of asthmatic airway inflammation (James A. Curr Opin Pulm Med 11(1):1-6 (2005)). Peripheral blood (PB) eosinophilia is a risk factor for relapse of acute asthma (Janson C and Herala M. Resp Med 86(2):101-104 (1992)). In subjects with peripheral blood eosinophilia, the risk of dying from asthma was 7.4 (confidence interval, 2.8-19.7) times greater than in those without eosinophilia (Ulrik C and Fredericksen J. Chest 108:10-15 (1995)). Necropsy results have identified 2 distinct pathogenic inflammatory mechanisms of fatal asthma (Restrepo R and Peters J. Curr Opin Pulm Med 14: 13-23 (2008)). A neutrophilic infiltrate is more prominent in those dying suddenly (approximately within 2 hours on onset of symptoms) while an eosinophilic infiltrate is more common in those dying from more protracted asthma crises. Sputum and blood eosinophils can also be increased in patients presenting to the ED with rapid onset of asthma symptoms (Bellido-Casado J, et al. Arch Bronconeumol 46(11): 587-93 (2010)). Therapies that target eosinophils lead to a reduction in the number and severity of asthma exacerbations as compared to the use of clinical guidelines (Green R, et al. Lancet 360:1715-21 (2002); Haldar P, et al. NEJM 360:973-84 (2009)).

Benralizumab (MEDI-563) is a humanized monoclonal antibody (mAb) that binds to the alpha chain of the interleukin-5 receptor alpha (IL-5Rα), which is expressed on eosinophils and basophils inducing apoptosis via antibody-dependent cell cytotoxicity. A single intravenous (IV) dose of benralizumab administered to adults with mild asthma provoked prolonged PB eosinopenia likely due to the effects on eosinophil/basophil bone marrow progenitors that express the target (Busse W, et al. JACI 125: 1237-1244 e2 (2010)). Benralizumab does not affect other cell lineages in the bone marrow or periphery. (Kolbeck R, et al. JACI 125:1344-53 (2010)).

Previous studies have demonstrated that an outpatient strategy focused on reducing eosinophils in the sputum reduces the number of subsequent asthma exacerbations (Green R, et al. Lancet 360:1715-21 (2002); Haldar P, et al. NEJM 360:973-84 (2009)).

Thus given the high unmet need of reducing the frequency and severity of acute exacerbations of asthma and that some subjects with acute asthma have an eosinophilic component, we evaluated the effect of intravenous benralizumab on the subsequent asthma exacerbation rate in adult subjects who required an ED visit and/or hospitalization for asthma and were discharged home after receiving current standard of care medications.

### BRIEF SUMMARY

In certain aspects, a method of reducing the number and severity of acute exacerbations of asthma in an asthma patient is provided, where the method includes administering to a patient with a history of acute exacerbations of asthma an effective amount of an anti-interleukin-5 receptor (IL-5R) antibody or antigen-binding fragment thereof. In certain aspects of the provided method, the patient's severe acute exacerbations are characterized by one or more of the following symptoms: (a) wheezing; (b) dyspnea; (c) a forced expiratory volume in one second (FEV₁) no more than 60%, 70%, or 80% of predicted value; (d) a peak expiratory flow (PEF) of no more than 60%, 70%, or 80% of predicted value; (e) coughing; or (f) two or more of these symptoms. In certain aspects, the patient's exacerbations are refractory to bronchodilator treatments.

In certain aspects, the provided method reduces the number of acute exacerbations over a 12-week period following administration of the antibody or antigen-binding fragment thereof, as compared to the number of exacerbations expected according to the patient's history, for example, the number of recurring exacerbations is reduced by at least 40% over the 12-week period. In certain aspects the provided method reduces the number of acute exacerbations over a 24-week period following administration of the antibody or antigen-binding fragment thereof, as compared to the number of exacerbations expected according to the patient's history.

In certain aspects, the provided method reduces the severity of one or more acute exacerbations, as compared to the severity of exacerbations expected according to the patient's history. For example, the provided method can reduce the number of exacerbations requiring an Emergency Department visit by at least 50% over a 12-week period, or reduce the number of exacerbations requiring hospitalization by at least 40% over a 12-week period. In certain aspects, the length of any required hospitalizations is reduced. In certain aspects, the number of hospitalizations requiring ICU admission is reduced.

In certain aspects of the provided method, the antibody or antigen binding fragment thereof is a monoclonal antibody, e.g., a chimeric antibody, a humanized antibody, or a fully human antibody or antigen-binding fragment thereof. In certain aspects, the antibody or antigen-binding fragment thereof specifically binds to the IL-5R α chain. In certain aspects, the antibody or antigen-binding fragment thereof further includes a constant region, e.g., an immunoglobulin Fc region. In certain aspects an immunoglobulin Fc region is altered in a manner that increases effector function, e.g., the immunoglobulin Fc region has reduced levels of fucose, or no fucose. In certain aspects an immunoglobulin Fc region includes amino acid substitutions that yield increased effector function, for example, including one or more of the following amino acid substitutions: 332E. 239D and 330L. as numbered by the EU index as set forth in Kabat.

In certain aspects the antibody or antigen-binding fragment thereof binds to the same IL-5Rα epitope as benralizumab. In certain aspects the antibody or antigen-binding fragment thereof is benralizumab or an antigen-binding fragment thereof.

In certain aspects the anti-IL-5R antibody or antigen-binding fragment thereof is administered as a single dose. In certain aspects the anti-IL5R antibody or antigen-binding fragment thereof is administered as two or more doses which can be spaced, for example, at least five (5) weeks apart or at least twelve (12) weeks apart.

In certain aspects single dose or first dose of the anti-IL-5R antibody or antigen-binding fragment thereof is administered to the patient within 7 days of an acute exacerbation of asthma. In certain aspects the anti-IL5R antibody or antigen-binding fragment thereof is administered at a dose of between about 0.1 mg/kg and 2 mg/kg per dose. In certain aspects the dose can be, for example, 0.3 mg/kg or 1 mg/kg.

In certain aspects the anti-IL5R antibody or antigen-binding fragment thereof is administered parenterally, e.g., intravenously. In certain aspects the anti-IL5R antibody or antigen-binding fragment thereof is administered in addition to corticosteroid therapy.

In certain aspects of the provided method, the patient's acute exacerbations are severe. In certain aspects, a course of systemic corticosterioids given in the hospital on discharge is not completely effective at reducing eosinophil count in the patient. In certain aspects the patient is not fully compliant with standard post-hospital therapy.

In certain aspects of the provided method, the anti-IL5R antibody or antigen-binding fragment thereof depletes eosinophil count independent of compliance with standard therapy. In certain aspects, the patient presents with one or more of the following characteristics: an elevated circulating eosinophil count, an elevated eosinophil count in induced sputum, an elevated eosinophil cationic protein level, an elevated eosinophil-derived neurotoxin level or a combination of the recited characteristics. In certain aspects the patient presents with one or more of the following characteristics: a normal circulating eosinophil count, a normal eosinophil count in induced sputum, a normal eosinophil cationic protein level, a normal eosinophil-derived neurotoxin level or combination of the recited characteristics.

### BRIEF DESCRIPTION OF THE DRAWINGS/FIGURES

FIG. 1A is a schematic of the design of the clinical study outlined in Examples 1 and 2. FIG. 1B is a schematic of subject disposition. Analysis was performed on evaluable subjects and on Study Days 84 and 168. Evaluable subjects were defined as those subjects followed through at least Study Day 42.

FIG. 2 shows the cumulative number of adjudicated asthma exacerbations for each treatment group to 24 weeks.

FIG. 3 shows the cumulative number of adjudicated asthma exacerbations resulting in hospitalization to 24 weeks.

FIG. 4 shows the effects of treatment with benralizumab on eosinophil counts.

### DETAILED DESCRIPTION

It is to be noted that the term “a” or “an” entity refers to one or more of that entity; for example, “an anti-IL-5α antibody” is understood to represent one or more anti-IL-5α antibodies. As such, the terms “a” (or “an”), “one or more,” and “at least one” can be used interchangeably herein.

Provided herein are methods for reducing the number and severity of recurrences of acute exacerbations of asthma in patients with a history of such recurrences. The methods provided include administering to the patient an effective amount of an antibody which specifically binds to the interleukin-5 receptor, for example to the alpha subunit of interleukin-5 receptor (IL-5Rα), or an antigen-binding fragment thereof. One aspect provides a method of reducing the number and severity of acute exacerbations of asthma in an asthma patient, including administering to a patient with a history of previous severe acute exacerbations of asthma an effective amount of an anti-interleukin-5 receptor (IL-5R) antibody or antigen-binding fragment thereof. In certain aspects, the antibody specifically binds to IL-5Rα. In certain aspects, the patient has presented to the emergency room or is hospitalized with a severe asthma exacerbation.

Anti-IL-5Rα antibodies or antigen-binding fragments thereof for use in the methods provided herein include, but are not limited to, monoclonal antibodies, synthetic antibodies, multispecific antibodies (including bi-specific antibodies), human antibodies, humanized antibodies, chimeric antibodies, single-chain Fvs (scFv) (including bi-specific scFvs). single chain antibodies. Fab fragments, F(ab″) fragments, disulfide-linked Fvs (sdFv), and epitope-binding fragments of any of the above. In particular, antibodies for use in the methods provided herein include immunoglobulin molecules and immunologically active portions of immunoglobulin molecules Immunoglobulins for use in the methods provided herein can be of any type (e.g., IgG, IgE, IgM, IgD. IgA and IgY), class (e.g. IgG 1, IgG2, IgG3, IgG4, IgA 1 and IgA2) or subclass of immunoglobulin molecule.

Nonlimiting examples of antibodies (or fragments thereof) for use in the methods provided herein can be found in U.S. Patent Application Publication No. US 2010/0291073 A1, the disclosure of which is incorporated herein by reference in its entirety. In a further aspect, an anti-IL-5Rα antibody or antigen-binding fragment thereof for use in the methods provided herein includes any one of the amino acid sequence of SEQ ID NO: 1-4. In a specific aspect, an anti-IL-5Rα antibody or antigen-binding fragment thereof for use in the methods provided herein includes the amino acid sequences SEQ ID NO: 1 and 3. In a specific aspect, an anti-IL-5Rα antibody or antigen-binding fragment thereof for use in the methods provided herein includes the amino acid sequences SEQ ID NO: 2 and 4.

Anti-IL-5Rα antibodies or antigen-binding fragments thereof for use in the methods provided herein can be engineered to possess increased effector function. Nonlimiting examples of methods for increasing effector function can be found in U.S. Pat. Nos. 5,624,821, 6,602,684, 7,029,872, U.S. Patent Application Publication Nos. 2006/0067930A1, 2005/0272128A1, 2005/0079605A1, 2005/0123546A1, 2004/0072290A1, 2006/0257399A1, 2004/0261148A1, 2007/0092521, 2006/0040325A1, and 2006/0039904A1, and International Patent Application Publication Nos. WO 04/029207, WO03/011878, WO05/044859, WO06/071856, and WO06/071280. Methods of engineering Fc regions of antibodies so as to alter effector functions are known in the art (e.g., U.S. Patent Publication No. 20040185045 and PCT Publication No. WO 2004/016750, both to Koenig et al., which describe altering the Fc region to enhance the binding affinity for FcγRIIB as compared with the binding affinity for FCγRIIA; see, also, PCT Publication Nos. WO 99/58572 to Armour et al., WO 99/5 1642 to Idusogie et al., and U.S. Pat. No. 6,395,272 to Deo et al.; the disclosures of which are incorporated herein in their entireties). Methods of modifying the Fc region to decrease binding affinity to FcγRI IB are also known in the art (e.g., U.S. Patent Publication No. 20010036459 and PCT Publication No. WO 01/79299, both to Ravetch et al., the disclosures of which are incorporated herein in their entireties). Modified antibodies having variant Fc regions with enhanced binding affinity for FcγRIIIA and/or FcγRIIA as compared with a wildtype Fc region have also been described (e.g., PCT Publication Nos. WO 2004/063351, to Stavenhagen et al., the disclosure of which is incorporated herein in its entirety).

Antibody effector function can also be modified through the generation of antibodies with altered glycosylation patterns. For example, an antibody can be made that has an altered type of glycosylation. such as an afucosylated/hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, EP 1, 176, 195 by Hanai et al. describes a cell line with a functionally disrupted FUT8 gene, which encodes a fucosyl transferase, such that antibodies expressed in such a cell line exhibit hypofucosylation. PCT Publication WO03/035835 by Presta describes a variant CHO cell line, Led 3 cells, with reduced ability to attach fucose to Asn(297)-linked carbohydrates, also resulting in hypofucosylation of antibodies expressed in that host cell (see also Shields, R. L. et al. (2002) J. Biol. Chem. 277:26733-26740). PCT Publication WO 99/54342 by Umana et al. describes cell lines engineered to express glycoprotein-modifying glycosyl transferases (e.g., beta(1,4)-N-acetylglucosaminyltransferase I II (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

In certain aspects, altered effector functions for an anti-IL-5Rα antibodies or antigen-binding fragments thereof for use in the methods provided herein are described in U.S. Patent Application Publication No. US 2008/0095765 A1, which is incorporated herein by reference in its entirety.

Basic immunoglobulin structures in vertebrate systems are relatively well understood. See, e.g., Harlow et al. (1988) Antibodies: A Laboratory Manual (2nd ed.; Cold Spring Harbor Laboratory Press).

In one aspect, an anti-IL-5Rα antibody or antigen-binding fragment thereof for use in the methods provided herein specifically binds to the same epitope as benralizumab. In a specific aspect, the antibody is benralizumab or an antigen-binding fragment thereof. In a further specific aspect, an anti-IL-5Rα antibody or antigen-binding fragment thereof for use in the methods provided herein specifically binds to the same epitope as benralizumab provided that the antibody is not benralizumab.

In one aspect, an anti-IL-5Rα antibody or antigen-binding fragment thereof for use in the methods provided herein specifically binds to an epitope within residues 1-102 of SEQ ID NO:5. In a specific aspect, the antibody is benralizumab. In a further specific aspect, a an anti-IL-5Rα antibody or antigen-binding fragment thereof for use in the methods provided herein specifically binds to an epitope within residues 1-102 of SEQ ID NO:5 provided that the antibody is not benralizumab.

In one aspect, an anti-IL-5Rα antibody or antigen-binding fragment thereof for use in the methods provided herein specifically binds to an epitope within residues 40-67 of SEQ ID NO:5. In a specific aspect, the antibody is benralizumab. In a further specific aspect, an anti-IL-5Rα antibody or antigen-binding fragment thereof for use in the methods provided herein specifically binds to an epitope within residues 40-67 of SEQ ID NO:5 provided that the antibody is not benralizumab. In one aspect, an anti-IL-5Rα antibody or antigen-binding fragment thereof for use in the methods provided herein specifically binds to an epitope within residues 52-67 of SEQ ID NO:5. In a specific aspect, the antibody is benralizumab. In a further specific aspect, an anti-IL-5Rα antibody or antigen-binding fragment thereof for use in the methods provided herein specifically binds to an epitope within residues 52-67 of SEQ ID NO:5 provided that the antibody is not benralizumab.

In one aspect, an anti-IL-5Rα antibody or antigen-binding fragment thereof for use in the methods provided herein is an antibody that specifically binds to an epitope including residue 61 of SEQ ID NO:5. In a specific aspect, the antibody is benralizumab. In a further specific aspect, an anti-IL-5Rα antibody or antigen-binding fragment thereof for use in the methods provided herein specifically binds to an epitope including residue 61 of SEQ ID NO:5 provided that the antibody is not benralizumab.

In certain aspects the asthma patient is a human aged 12 years or older. In certain aspects, the patient has a history of recurrences of acute exacerbations, in certain aspects the acute exacerbations are classified as severe according to the Expert Panel Report 3: Guidelines for the Diagnosis and Management of Asthma, National Asthma Education and Prevention Program (2007) (“NAEPP Guidelines”), incorporated herein by reference in its entirety.

In certain aspects, the patient's acute exacerbations are characterized by one or more symptoms selected from the group consisting of a worsening of: (a) wheezing; (b) dyspnea; or (c) cough; and (d) a forced expiratory volume in one second (FEV₁) or peak expiratory flow (PEF) of no more than between about 60% and about 80% of predicted value of no more than about 70% of their predicted normal value after treatment with bronchodilators.

In certain aspects, the patient's acute exacerbations of asthma can require a visit to a hospital emergency department (ED), a hospital admission of 1, 2, 3, 4, 5, or more days, or even an intensive care unit admission of 1, 2, 3, 4, 5, or more days. In certain aspects, the patient's acute exacerbations of asthma can require a visit to a hospital emergency department (ED), a hospital admission of 1 or more days, or even an intensive care unit admission of 1 or more days. In certain aspects the patient is fully compliant, moderately compliant, or non-compliant with physician-recommended instructions and/or prescribed therapies, such as those noted in the NAEPP Guidelines.

In certain aspects, the patient can exhibit poor asthma control as manifested by asthma symptoms more than two times per week, daily, or as often as multiple times each day. In certain aspects the patient requires bronchodilator (e.g., short- or long-acting β2-agonists, anticholinergics, or theophylline) treatment more than twice per week, daily, or as often as multiple times per day. In certain aspects, the patient's exacerbations require multiple bronchodilator treatments to control, or are refractory to bronchodilator treatments.

In certain aspects, a patient presenting at a physician's office or ED with an acute exacerbation of asthma, e.g., a severe acute exacerbation of asthma is administered a single dose of an anti-IL-5Rα antibody or antigen-binding fragment thereof. Given the ability of anti-IL-5Rα antibodies or antigen-binding fragments thereof, e.g., benralizumab, to reduce or deplete eosinophil counts for up to 12 weeks or more (see US 2010/0291073), an anti-IL-5Rα antibody or antigen-binding fragment thereof, e.g., benralizumab can be administered only once or infrequently while still providing benefit to the patient in reducing the frequency and severity of acute exacerbations. In further aspects the patient is administered additional follow-on doses. Follow-on doses can be administered at various time intervals depending on the patient's age, weight, ability to comply with physician instructions, clinical assessment, eosinophil count (blood or sputum eosinophils or eosinophilic cationic protein (ECP) measurement), or and other factors, including the judgment of the attending physician. Evaluations can include, for example, assessments of disease activity (spirometry, use of concomitant rescue medications, need to add inhaled steroids, and the Physician's Global Assessment [PGA]); patient-reported outcomes, e.g., asthma control questionnaire (ACQ) and asthma quality of life questionnaire (AQLQ); healthcare resource utilization and economics; safety assessments, e.g., adverse event and serious adverse event (SAE) evaluation, physical examination, vital signs, serum chemistry, hematology, urinalysis, and measurement of eosinophilic cationic protein (ECP), IL-6, and C-reactive protein (CRP); pharmacokinetics (PK), and immunogenicity. In addition, samples can be analyzed for eosinophil-derived proteins such as major basic protein (MBP) and eosinophil-derived neurotoxin (EDN); plasma eotaxin levels; and measurement of interleukins. The intervals between doses can be every five weeks, every 6 weeks, every 8 weeks, every 10 weeks, every 12 weeks, or longer intervals. In certain aspects the intervals between doses can be every 12 weeks. In certain aspects, the single dose or first dose is administered to the asthma patient shortly after the patient presents with an acute exacerbation, e.g., a mild, moderate or severe exacerbation. For example, the single or first dose of an anti-IL-5Rα antibody or antigen-binding fragment thereof, e.g., benralizumab can be administered during the presenting clinic or hospital visit, or in the case of very severe exacerbations, within 1, 2, 3, 4, 5, 6, 7, or more days, e.g., 7 days, of the acute exacerbation, allowing the patient's symptoms to stabilize prior to administration of benralizumab.

The amount of an anti-IL-5Rα antibody or antigen-binding fragment thereof, e.g., benralizumab to be administered to the patient will depend on various parameters such as the patient's age, weight, clinical assessment, eosinophil count (blood or sputum eosinophils, eosinophilic cationic protein (ECP) measurement or eosinophil derived neurotoxin (EDN) measurement), or and other factors, including the judgment of the attending physician. In certain aspects, the dosage or dosage interval is not dependent on the sputum eosinophil level. In certain aspects the patient is administered one or more doses of an anti-IL-5Rα antibody or antigen-binding fragment thereof, e.g., benralizumab, where the dose is between about 0.01 mg/kg and 2.0 mg/kg, for example between about 0.03 mg/kg and about 0.1 mg/kg, or between about 0.3 mg/kg and 1 mg/kg. In certain specific aspects the patient is administered one or more doses of an anti-IL-5Rα antibody or antigen-binding fragment thereof, e.g., benralizumab, where the dose is about 0.03 mg/kg, 0.1 mg/kg, 0.3 mg/kg, or 1.0 mg/kg.

In certain aspects, administration of an anti-IL-5Rα antibody or antigen-binding fragment thereof, e.g., benralizumab according to the methods provided herein is through parenteral administration. For example, the anti-IL-5Rα antibody or antigen-binding fragment thereof, e.g., benralizumab can be administered by intravenous infusion, or by subcutaneous injection.

In certain aspects, an anti-IL-5Rα antibody or antigen-binding fragment thereof, e.g., benralizumab is administered according to the methods provided herein in combination or in conjunction with additional asthma therapies. Such therapies include, without limitation, inhaled corticosteroid therapy, long- or short-term bronchodilator treatment, oxygen supplementation, or other standard therapies as described, e.g., in the NAEPP Guidelines. In certain aspects, use of the methods provided herein, i.e., administration of an anti-IL-5Rα antibody or antigen-binding fragment thereof, e.g., benralizumab to an asthma patient with a history of acute exacerbations serves as adjunct therapy in situations of poor compliance with standard forms of asthma management.

The methods provided herein can significantly reduce the number and severity of acute exacerbations of asthma. Reduction of frequency and severity can be measured based on the expected frequency and severity of exacerbations predicted based on a large patient population, or based on the individual patient's history of exacerbations. In certain aspects, the patient population is those patients who had more frequent and intense exacerbations (e.g., requiring urgent, unscheduled care, hospitalization, or ICU admission), or patients who had ≧2 exacerbations requiring oral systemic corticosteroids in the past year.

In certain aspects, use of the methods provided herein, i.e., administration of an anti-IL-5Rα antibody or antigen-binding fragment thereof, e.g., benralizumab to an asthma patient with a history of acute exacerbations, reduces the number of acute exacerbations experienced by the patient over a 12-week period following administration of the antibody or antigen-binding fragment thereof, as compared to the number of exacerbations expected according to the patient's history, or as compared to the average number of exacerbations expected in a comparable population of patients over the same time period. In certain aspects, the patient can receive follow on doses of an anti-IL-5Rα antibody or antigen-binding fragment thereof, e.g., benralizumab at periodic intervals, e.g., every 6 weeks, every 8 weeks, every 12 weeks, or as scheduled based on patient's age, weight, ability to comply with physician instructions, clinical assessment, eosinophil count (blood or sputum eosinophils or eosinophilic cationic protein (ECP) measurement), or and other factors, including the judgment of the attending physician. Use of the methods provided herein can reduce the frequency of recurring acute exacerbations by 10%, 20%, 30%, 40%, 50%, 75% or 100% over the 12-week period. In certain aspects, use of the methods provided herein can reduce the frequency of recurring acute exacerbations by 50%, and the frequency of severe exacerbations (requiring hospitalization) by 60%.

In other aspects, use of the methods provided herein, i.e., administration of an anti-IL-5Rα antibody or antigen-binding fragment thereof, e.g., benralizumab to an asthma patient with a history of acute exacerbations, reduces the number of acute exacerbations experienced by the patient over a 24-week period following administration of the antibody or antigen-binding fragment thereof, as compared to the number of exacerbations expected according to the patient's history, or as compared to the average number of exacerbations expected in a comparable population of patients over the same time period. In certain aspects, the patient can receive follow on doses of an anti-IL-5Rα antibody or antigen-binding fragment thereof, e.g., benralizumab at periodic intervals, e.g., every 6 weeks, every 8 weeks, every 12 weeks, or as scheduled based on patient's age, weight, ability to comply with physician instructions, clinical assessment, eosinophil count (blood or sputum eosinophils or eosinophilic cationic protein (ECP) measurement), or and other factors, including the judgment of the attending physician. In certain aspects, the interval is every 12 weeks. Use of the methods provided herein can reduce the frequency of recurring acute exacerbations by 10%, 20%, 30%, 40%, 50%, 75% or 100% over the 24-week period.

In certain aspects, use of the methods provided herein, i.e., administration of an anti-IL-5Rα antibody or antigen-binding fragment thereof, e.g., benralizumab to an asthma patient with a history of acute exacerbations reduces the severity of one or more acute exacerbations, as compared to the severity of exacerbations expected according to the patient's history, or as compared to the average severity of exacerbations expected in a comparable population of patients. In certain aspects, the patient can receive follow on doses of an anti-IL-5Rα antibody or antigen-binding fragment thereof, e.g., benralizumab at periodic intervals, e.g., every 6 weeks, every 8 weeks, every 12 weeks, or as scheduled based on patient's age, weight, ability to comply with physician instructions, clinical assessment, eosinophil count (blood or sputum eosinophils or eosinophilic cationic protein (ECP) measurement), or and other factors, including the judgment of the attending physician.

For example, in some aspects the number of acute exacerbations experienced by the patient requiring an Emergency Department visit can be reduced by 10%, 20%, 30%, 40%, 50%, 75% or 100% over a 12-week, 24-week, or longer period over the number of hospital admissions expected according to the patient's history, or as compared to the average number of hospital admissions expected in a comparable population of patients. In certain aspects the number of acute exacerbations experienced by the patient requiring an Emergency Department visit can be reduced by about 50%. In other aspects the number of acute exacerbations requiring hospital admission is reduced by 10%, 20%, 30%, 40%, 50%, 75% or 100% over a 12-week, 24-week, or longer period over the number of hospital admissions expected according to the patient's history, or as compared to the average number of hospital admissions expected in a comparable population of patients. In certain aspects the number of acute exacerbations requiring hospital admission is reduced by about 50%. Furthermore, in those acute exacerbations requiring hospital admission, use of the methods provided herein can reduce the length of the hospitalization expected according to the patient's history, or as compared to the average length of hospitalization expected in a comparable population of patients, e.g., the length of hospitalization required can be reduced by one day, two days, three days, four days, or more over that expected according to the patient's history, or as compared to the average expected in a comparable population of patients.

Similarly, use of the methods provided herein, i.e., administration of an anti-IL-5Rα antibody or antigen-binding fragment thereof, e.g., benralizumab to an asthma patient with a history of acute exacerbations can reduce the number of hospitalizations requiring ICU admission, and the length of any ICU stays.

In one aspect, use of the methods provided herein, i.e., administration of an anti-IL-5Rα antibody or antigen-binding fragment thereof, e.g., benralizumab to an asthma patient with a history of acute exacerbations can reduce the eosinophil count in the patient, either in serum or in induced sputum, measured during or after the onset of subsequent exacerbations. Circulating eosinophil count or induced sputum eosinophil count can be assessed using any methods known to one of skill in the art, for example, but not limited to histology, flow cytometry. Circulating eosinophil count or induced sputum eosinophil count can be measured by any one of the commercially available kits. In certain aspects, use of the methods provided herein, i.e., administration of an anti-IL-5Rα antibody or antigen-binding fragment thereof, e.g., benralizumab to an asthma patient with a history of acute exacerbations does not appreciably affect the circulating or induced sputum eosinophil counts measured during or after the onset of subsequent exacerbations. In certain aspects, asthma patient with a history of acute exacerbations does not have appreciably elevated circulating or induced sputum eosinophil counts at baseline, and thus a reduction in eosinophil counts cannot be measured.

In certain aspects, the asthma patient has an absolute circulating eosinophil count of between about 0 and about 350 cells/μl prior to the administration of an anti-IL-5Rα antibody or antigen-binding fragment thereof, e.g., benralizumab. In specific aspects, the asthma patient has an absolute circulating eosinophil count of about 40, about 70, or about 150 cells/μl prior to the administration of an anti-IL-5Rα antibody or antigen-binding fragment thereof, e.g., benralizumab. In certain aspects, the asthma patient has an absolute circulating eosinophil count of between about 0 and about 200 cells/μl at a time point at least one week, at least two weeks, at least 6 weeks, at least 8 weeks, or at least 12 weeks following the administration of an anti-IL-5Rα antibody or antigen-binding fragment thereof, e.g., benralizumab. In specific aspects, the asthma patient has an absolute circulating eosinophil count about 1, about 3, about 5, about 10, about 30, about 50, about 60, or about 75 cells/μl at a time point at least one week, at least two weeks, at least 6 weeks, at least 8 weeks, or at least 12 weeks following the administration of an anti-IL-5Rα antibody or antigen-binding fragment thereof, e.g., benralizumab. In certain aspects, the asthma patient has no detectable circulating eosinophils following the administration of an anti-IL-5Rα antibody or antigen-binding fragment thereof, e.g., benralizumab out to about 8 weeks, about 10 weeks, about 12 weeks, about 18 weeks, or about 24 weeks. In certain aspects, the asthma patient's absolute circulating eosinophil count prior to the administration of an anti-IL-5Rαantibody or antigen-binding fragment thereof, e.g., benralizumab is reduced by about 10%, 20%, 30%, 40%, 50%, 60%, 70%, 80%, 90% or 100% at a time point at least one week, at least two weeks, at least 6 weeks, at least 8 weeks, or at least 12 weeks following the administration of an anti-IL-5Rα antibody or antigen-binding fragment thereof, e.g., benralizumab.

In one aspect, the disease indicator or symptom is percent (%) eosinophil in induced sputum. Percent eosinophil in induced sputum can be assessed using any methods known to one of skill in the art, for example, but not limited to the methods described in Belda et al. (2000) Am J Respir Grit Care Med 161:475-478. Percent eosinophil in induced sputum can be determined by any one of the commercially available kits.

### EXAMPLES

### Example 1

### Patients and Methods

### (a) Subjects

Subjects in this study were 18 to 60 years of age with a physician diagnosis of asthma for a minimum of 2 years duration and met National Heart Lung and Blood Institute (NHLBI) guidelines for persistent asthma in the previous 3 months. Subjects were recruited from patients who were evaluated in the emergency department (“ED”) for an asthma exacerbation that had been ongoing for a minimum of 2 hours at presentation. Eligible patients must have received at least 2 treatments with inhaled bronchodilators either in the ED or in the emergency medical system (EMS) with an incomplete clinical response which was defined as a forced expiratory volume in one second (FEV₁) or peak expiratory flow (PEF) of not more than 70% predicted value. In addition, these patients must also have experienced at least one other asthma exacerbation requiring an urgent care visit in the past 12 months. Subjects were allowed to be active tobacco smokers with a total exposure of no more than 20 pack-years however a physician diagnosis of chronic obstructive pulmonary disease (COPD) was not allowed. Other exclusions to participation in this study include another acute illness at entry in to the study, fever >38.6° C., aspirin-induced asthma attack, anaphylactic/anaphylactoid reaction presenting with bronchospasm, acute inhalational exposure, symptoms of or exposure to parasitic infections, and immunodeficiency. All subjects provided written informed consent prior to participation in this study.

### (B) Design of the Study

The study was a phase 2, multi-center, randomized, double-blind, placebo-controlled, parallel group study conducted at 15 sites across US and Canada between February 2009 and March 2011 (ClinicalTrials.gov number: NCT00768079).

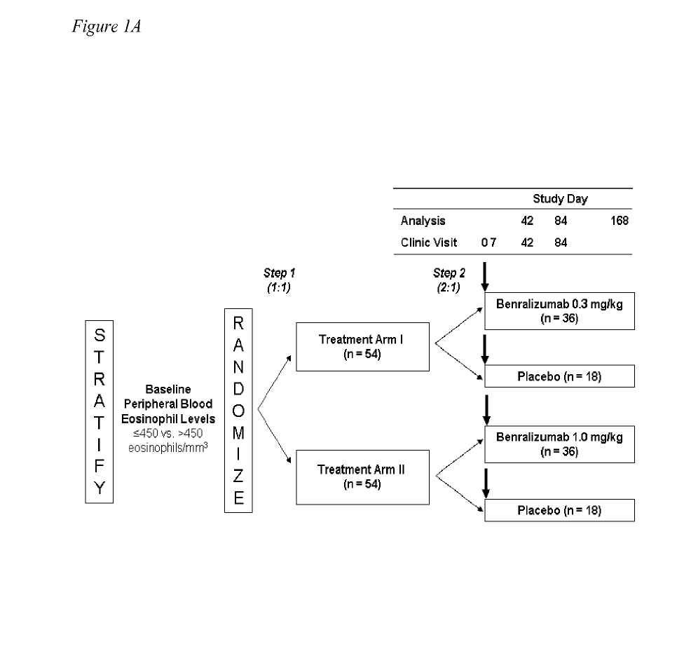

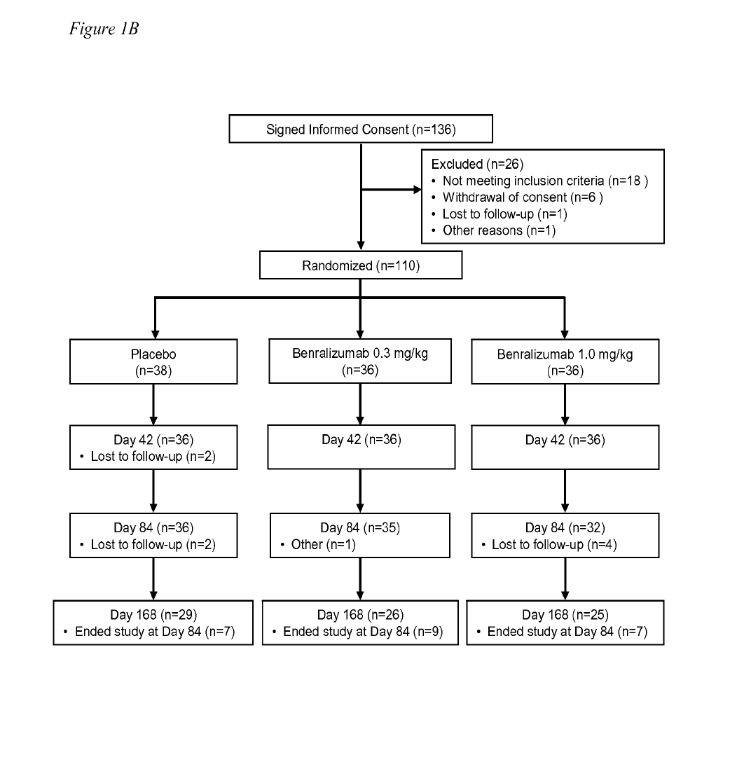

The design of the study is outlined in FIG. 1A. Patients who consented to participate in this study had a screening eosinophil count performed and dosing with either placebo or benralizumab could be performed up to 7 days later. Prior to treatment, subjects were required to be clinically stable and to have an FEV₁ between 30-70% predicted. Subjects were stratified by baseline eosinophil count of greater or less than 450 eosinophils/mm³, randomized into one of two treatment arms for benralizumab (0.3 mg/kg and 1.0 mg/kg), and then further randomized at a 2:1 ratio into active treatment or placebo. Dose selection was based upon the expected duration of eosinopenia, which was approximately 84 days for 0.3 mg/kg and >84 days for 1.0 mg/kg. Placebo and benralizumab doses were identical in appearance (Supplementary Appendix FIG. 1). Randomization was performed by an interactive voice/web response system. Upon discharge from the ED or hospital, all subjects received a minimum of 40 mg/day of prednisone or equivalent for at least 7 days and were given a prescription for inhaled corticosteroids. Subjects were required to be clinically stabilized and demonstrate an FEV₁≧30% of predicted normal prior to dosing. Subjects were dosed up to 7 days after a subject's qualifying asthma exacerbation with either placebo or benralizumab administered as a single IV infusion over at least 30 minutes.

Subjects were followed for a total of 168 days after dosing. There were scheduled clinic visits at 7, 42 and 84 days following dosing. In addition, subjects had telephone calls at 28, 63, 112, 140, and 168 days. Study measurements included FEV₁, Asthma Control Questionnaire (ACQ), and Asthma Quality of Life Questionnaire (AQLQ), (See, e.g., Juniper E F, et al. Chest 115(5):1265-70 (1999), and Juniper E F, et al. Eur Respir J. 14(4):902-7 (1999)), use of rescue medications, physician assessment of health status, healthcare resource utilization, safety assessments, pharmacokinetics, and immunogenicity.

The initial qualifying asthma exacerbation was managed in accordance with the NAEPP Guidelines by the treating healthcare provider. Upon discharge from the ED or hospital, all subjects received a minimum 7-day supply of 40 mg/day prednisone or equivalent and a prescription for inhaled corticosteroids (ICS). Subjects were dosed up to 7 days after a subject's qualifying asthma exacerbation, with either placebo or benralizumab was administered as a single IV infusion over at least 30 minutes.

The primary efficacy outcome was the proportion of subjects with ≧1 exacerbation at week 12. Secondary outcomes included the proportion of subjects with an exacerbation at weeks 4 and 24, safety assessments, asthma symptom changes, health-related quality of life, lung function, eosinophil counts, and healthcare resource utilization. The time-weighted rate of exacerbations at week 12 was added as an efficacy endpoint prior to unblinding the study and data analysis. In this study, asthma exacerbations were defined as either 1) an increase of asthma symptoms that did not resolve within 2 hours after the use of rescue albuterol or corticosteroids and required an unscheduled medical visit or 2) during a scheduled study visit, the subject showed acute symptoms of asthma and a reduction of ≧20% in PEF or FEV₁, which in the opinion of the investigator required treatment. For each asthma exacerbation date of onset, date of visit to healthcare provider or ED, treatment received and resolution date were collected. A period of 7 days of clinical stability following resolution of an exacerbation was required between exacerbations. Reported exacerbations were adjudicated in a blinded fashion to determine whether a reported exacerbation met the protocol definition. An exacerbation that occurred within 7 days of the previous exacerbation was counted as a single asthma exacerbation in accordance with the protocol definition.

### (C) Safety Assessments

Adverse events were monitored following administration of placebo or benralizumab through Week 24. Other assessments included physical examination, vital sign monitoring, and laboratory measurements.

### (D) Statistical Analysis

Sample size was calculated for the proportion of subjects with at least one asthma exacerbation using Fisher's exact test with a two-sided alpha level of 0.05 for testing the difference between the combined benralizumab treatment groups and the placebo group. With 108 subjects (36 in the placebo group and 72 in the combined benralizumab group), the study had an 80% power to detect a 50% difference in exacerbation rate. Assumptions included a 60% asthma exacerbation rate for the placebo group at Week 12 and an alpha level of 0.05.

Reported exacerbations were adjudicated in a blinded fashion to determine whether an exacerbation met the protocol definition. If an onset date of an asthma exacerbation occurred within 7 days of the end date of a previous exacerbation, the exacerbation was considered a continuation of the previous exacerbation.

The weighted asthma exacerbation/hospitalization rate over a given period equaled (total number of exacerbations/hospitalizations)/(total duration of person-year follow-up). Person-year follow-up for each subject equaled (number of days between first dose and last contact or cut-off, whichever came first)/365.25 days. The Poisson model with an offset option compared the weighted asthma exacerbation/hospitalization rate between the combined treatment and placebo groups. The reduction in rate was calculated by taking exponentiation of the coefficient for the combined treatment group in the Poisson regression model.

The evaluable population and the intent-to-treat population were used in the efficacy analyses and all subjects who received at least one dose of investigational product were included in the safety analysis. Subjects were considered evaluable if they received the investigational product and were followed according to the protocol through Study Day 42. Missing data was treated as missing without data imputation.

Before the first interim analysis, the weighted asthma exacerbation rate through study week 12 was added as one of the primary efficacy endpoints and was documented in the interim unblinding analysis plan.

### (E) Asthma Exacerbation Adjudication

Asthma exacerbations were adjudicated in a blinded fashion by the sponsor medical monitor to determine whether the reported exacerbation met the protocol definition—particularly regarding the requirements for an unscheduled medical visit and for 7 days of stability between exacerbations. A second person, not a member of the study team, conducted an independent adjudication of the data. Discrepancies were resolved by consensus agreement between the two reviewers.

Asthma exacerbations that were managed at home without healthcare provider contact were not counted as an exacerbation regardless of the treatment received. Asthma exacerbations followed closely by a subsequent exacerbation within 7 days of resolution of the previous exacerbation were counted as a single exacerbation. For instance, a subject may experience an exacerbation and go to the emergency department (ED) be treated and discharged. If the subject then relapsed and returned to the ED within 7 days for additional treatment this would be treated as a single asthma exacerbation requiring ED care. If this same subject had returned and been admitted to the hospital within 7 days, this would be treated as a single asthma exacerbation requiring hospital care. In some instances, subjects experienced multiple (>2) events, which counted as single exacerbation. This adjudication process was not applied to the healthcare resource utilization analysis as each event was counted separately.

### Example 2

### Results

### (a) Enrollment and Baseline Characteristics

One hundred thirty-six (136) subjects with acute asthma exacerbations were screened and 110 were enrolled in the study. Two subjects in the placebo group were lost to follow up after dosing, and were not included in the evaluable population. One hundred and eight (108) subjects completed evaluations through day 42 and were considered evaluable (36 subjects/group) at the primary endpoint of 84 days (FIG. 1B). Overall, 80 (73%) of the 110 randomized subjects were followed for the entire 24 weeks. The demographics and baseline asthma characteristics of the study population are shown in Table 1. The three cohorts were comparable in respect to asthma history and asthma control at entry into the study. The majority of subjects in this study were obese with BMI>30. Asthma Control Questionnaire (ACQ) scores were high and Asthma Quality of Life Questionnaire (AQLQ) scores were low but not unexpected for patients upon presentation to the emergency department for acute asthma.

TABLE 1
Baseline characteristics for the intent to treat population

| | Benralizumab | | | |
|---|---|---|---|---|
| | Placebo | 0.3 mg/kg | 1.0 mg/kg | Total |
| Characteristic^{a} | n = 38 | n = 36 | n = 36 | n = 72 |
| Age, y | | | | |
| Mean | 35.9 | 37.9 | 34.8 | 36.3 |
| Range | 22-55 | 20-60 | 19-54 | 19-60 |
| Gender, No. | | | | |
| Male | 8 | 12 | 13 | 25 |
| Female | 30 | 24 | 23 | 47 |
| Race, No. | | | | |
| Black | 26 | 19 | 19 | 38 |
| White | 11 | 14 | 16 | 30 |
| Other | 1 | 3 | 1 | 4 |
| Body mass index, kg/m² | 34.7 (8.9) | 30.1 (8.1) | 30.8 (8.3) | 30.5 (8.2)^{b} |
| Smoking status | | | | |
| Ever smoked, % | 55.3 | 58.3 | 50.0 | 54.2 |
| Current smoker, % | 20.0 | 26.5 | 40.0 | 32.8 |
| Marijuana smoker, % | 13.2 | 16.7 | 33.3 | 25.0 |
| FEV₁ | | | | |
| At dosing date, L | 1.68 | 1.69 | 2.07 | 1.88^{b} |
| After BD treatment in | 56.1 | 53.1 | 63.3 | 58.1 |
| ED, % predicted | | | | |
| Eosinophil count, 10³/μL | 0.350 (0.525) | 0.259 (0.511) | 0.168 (0.220) | 0.213 (0.393) |
| Eosinophil count, | | | | |
| 10³/μL, n (%)^{c} | | | | |
| =0 | 12 (32) | 17 (47) | 13 (36) | 30 (42) |
| >0-≦0.3 | 13 (35) | 12 (33) | 16 (44) | 28 (39) |
| >0.3 | 12 (32) | 7 (19) | 7 (19) | 14 (19) |
| Asthma controller use at | | | | |
| entry, n (%).^{d} | | | | |
| ICS | 8 (22) | 9 (25) | 6 (17) | 15 (21) |
| ICS/LABA | 22 (61) | 20 (56) | 19 (53) | 39 (54) |
| LABA | 4 (11) | 5 (14) | 2 (6) | 7 (10) |
| LTRI | 12 (33) | 9 (25) | 8 (22) | 17 (24) |
| LAMA | 0 (0) | 2 (6) | 1 (3) | 3 (4) |
| Steroid bursts past 3 | | | | |
| months, No. | | | | |
| 0 | 13 | 11 | 11 | 22 |
| 1 | 9 | 9 | 11 | 20 |
| 2 | 2 | 10 | 8 | 18 |
| 3 | 6 | 3 | 4 | 7 |
| ≧4 | 8 | 3 | 2 | 5 |
| ED Visits past 12 months | | | | |
| Mean | 6.0 | 5.6 | 3.7 | 4.6 |
| Median | 3.0 | 2.0 | 2.0 | 2.0 |
| Range | 0-30 | 0-40 | 0-20 | 0-40 |
| Hospital admissions past | | | | |
| 12 months | | | | |
| Mean | 2.6 | 1.9 | 1.3 | 1.6 |
| Median | 1.5 | 1.0 | 1.0 | 1.0 |
| Range | 0-20 | 0-13 | 0-8 | 0-13 |
| ICU admissions ever | | | | |
| Mean | 2.5 | 3.2 | 3.0 | 3.1 |
| Median | 0.5 | 0.0 | 0.5 | 0.0 |
| Range | 0-50 | 0-80 | 0-30 | 0-80 |
| ACQ | 3.64 (0.83) | 3.72 (1.18) | 3.26 (1.27) | 3.49 (1.24) |
| AQLQ | 3.10 (0.90) | 3.18 (0.95) | 3.35 (1.11) | 3.27 (1.03) |

Abbreviations:
ACQ, Asthma Control Questionnaire;
AQLQ, Asthma Quality of Life Questionnaire;
BD, bronchodilator;
ED, emergency department;
ICS, inhaled corticosteroid;
FEV₁, forced expiratory volume in 1 second;
LABA, long-acting beta agonist;
LAMA, long-acting muscarinic agonist;
LTRI, leukotriene modifier;
ICU, intensive care unit;
^{a}Values are expressed as mean (SD) unless otherwise indicated.
^{b}P < 0.05 for differences among 3 groups by analysis of variance.
^{c}Blood samples for eosinophil counts were collected at screening when a subject presented to the ED and analyzed at the local lab. Placebo group n = 37.
^{d}Placebo group is based on the evaluable population (N = 36). Controller use at entry includes an asthma medication with onset at study day −7 or earlier.

Baseline demographic and clinical characteristics were similar between the groups except for BMI and post-treatment FEV1 at dosing date (Table 1). Systemic corticosteroid use for both the initial treatment and during the course of the study was comparable for all treatment groups (Table 2).

TABLE 2
Asthma treatment of study (evaluable)

Benralizumab

| | Placebo | 0.3 mg/kg | 1.0 mg/kg |
|---|---|---|---|
| Asthma Treatment | n = 36 | n = 36 | n = 36 |
| Systemic corticosteroids, | | | |
| total dose, mg | | | |
| Mean | 666 | 717 | 730 |
| SD | 824 | 1224 | 897 |
| Range | 80-4436 | 15-5909 | 60-3730 |
| Controller therapy, n (%) | | | |
| ICS | 17 (47) | 4 (11) | 11 (31)^{b} |
| ICS/LABA | 18 (50) | 17 (47) | 11 (31) |
| LABA | 1 (3) | 2 (6) | 2 (6) |
| LTRI | 7 (19) | 11 (31) | 3 (8) |
| LAMA | 0 (0) | 3 (8) | 4 (11) |

Abbreviations:
ICS, inhaled corticosteroid;
LABA, long-acting beta agonist;
LAMA, long-acting muscarinic agonist;
LTRI, leukotriene modifier.
^{a}Values are for the evaluable population during the conduct of the study (day 0 through day 84)
^{b}P < 0.05 for differences among 3 groups by the Fisher's exact test.

### (B) Efficacy

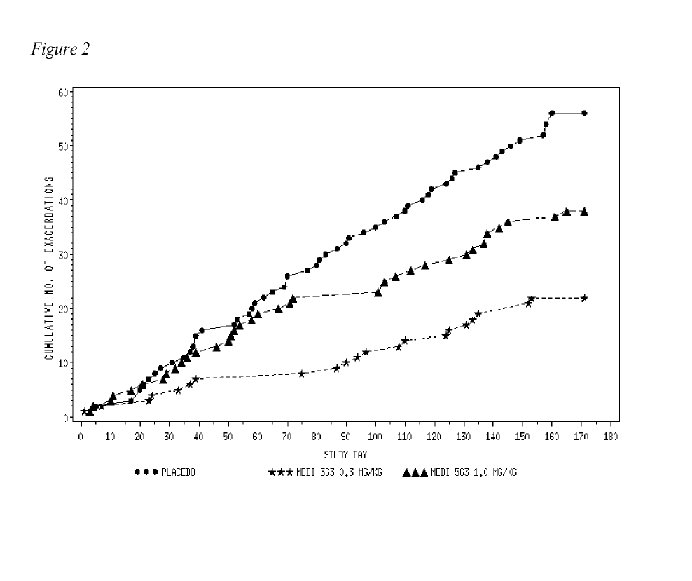

The proportion of subjects who experienced ≧1 asthma exacerbation through week 12 was 14/36 (38.9%) vs. 24/72 (33.3%; P=0.67) for the placebo vs. combined treatment groups, respectively. (Table 3) The proportion of subjects who experienced ≧2 asthma exacerbations through week 12 was 9/36 (25%) vs. 5/72 (6.9%; P=0.01) for the placebo vs. combined treatment groups, respectively. (data not shown) The number of exacerbations through week 12 was 31 from 36 evaluable subjects in the placebo group and 31 from 72 evaluable subjects in the combined treatment groups (FIG. 2). Compared to placebo, the asthma exacerbation rate was reduced by 49% (Table 3). The weighted asthma exacerbation rate (exacerbations/subject/year) was 3.59 (95% CI: 2.44-5.10) for placebo vs. 1.05 (95% CI 0.48-1.99; P=0.001) for 0.3 mg/kg with a relative risk of 3.43 (95% CI: 1.59-8.18), 2.61 (95% CI 1.63-3.95, P=0.250) for 1.0 mg/kg with a relative risk of 1.38 (95% CI: 0.77-2.50), and 1.82 (95% CI 1.24-2.59; P=0.007) for the combined treatment groups with a relative risk of 1.97 (95% CI: 1.16-3.36). Two of 62 adjudicated asthma exacerbations (1 each in the placebo and 0.3 mg/kg groups) were not treated with systemic corticosteroids for the exacerbation. Analysis of asthma exacerbations based upon peripheral blood eosinophil levels was not useful due to the number of subjects who had been exposed to systemic corticosteroids resulting in low or unmeasurable eosinophil levels at baseline. The number of subjects with high (or low) eosinophil counts was similar between the two treatment groups (Table 1). Exacerbation rates in treated subjects were similar for subjects who had eosinophil counts >0.3×103/μL (1.50 exacerbations/subject/year; 95% CI 0.49, 3.51) at screening compared to treated subjects with counts of ≦0.3×103/μL (1.90 exacerbations/subject/year; 95% CI 1.24-2.78) (Table 4).

TABLE 3
Primary outcome measures for the evaluable population

| | | Benralizumab | | | |
|---|---|---|---|---|---|
| Primary Outcome | Placebo | 0.3 mg/kg | 1.0 mg/kg | Total | |
| Measure | n = 36 | n = 36 | n = 36 | n = 72 | P Value |
| Subjects with ≧1_asthma | | | | | |
| exacerbation, n (%) | | | | | |
| Week 4 | 8 (22.2%) | 4 (11.1%) | 8 (22.2%) | 12 (16.7%) | .600^{a} |
| Week 12 | 14 (38.9%) | 9 (25.0%) | 15 (41.7%) | 24 (33.3%) | .670^{a} |
| Week 24 | 17 (47.2%) | 13 (36.1%) | 18 (50.0%) | 31 (43.1%) | .687^{a} |
| Subjects with_≧1 | | | | | |
| hospitalization, n (%) | | | | | |
| Week 4 | 4 (11.1%) | 3 (8.3%) | 2 (5.6%) | 5 (6.9%) | .478^{a} |
| Week 12 | 7 (19.4%) | 3 (8.3%) | 5 (13.9%) | 8 (11.1%) | .251^{a} |
| Week 24 | 10 (27.8%) | 6 (16.7%) | 7 (19.4%) | 13 (18.1%) | .319^{a} |
| Weighted asthma | 3.59 | 1.05 | 2.61 | 1.82 | .007^{b} |
| exacerbation rate | (2.44, 5.10) | (0.48, 1.99) | (1.63, 3.95) | (1.24, 2.59) | |
| through week 12 | | | | | |
| (95% CI) | | | | | |
| Weighted hospitalization | 1.62 | 0.35 | 0.95 | 0.65 | .022^{c} |
| rate through week 12 | (0.89, 2.72) | (0.07, 1.02) | (0.41, 1.87) | (0.32, 1.16) | |
| (95% CI) | | | | | |

^{a}P value by Fisher's Exact Test comparing benralizumab combined group to placebo.
^{b}P value by Poisson regression model without overdispersion comparing benralizumab combined group to placebo. P = .020 by Poisson regression model with overdispersion.
^{c}P value by Poisson regression model without overdispersion comparing benralizumab combined group to placebo. P = .019 by Poisson regression model with overdispersion.

TABLE 4
Exacerbation rates by eosinophil count for the evaluable populations

| | | Exacerbation | |
|---|---|---|---|
| Treatment Group | n | Rate (95% CI) | P Value |
| Eosinophil count ≦0.3 × | | | |
| 10³/μL | | | |
| Placebo | 23 | 4.53 (2.93, 6.69) | |
| Benralizumab 0.3 mg/kg | 29 | 1.01 (0.41, 2.08) | <.001 |
| Benralizumab 1.0 mg/kg | 29 | 2.81 (1.69, 4.39) | .117 |
| Benralizumab total | 58 | 1.90 (1.24, 2.78) | .002 |
| Eosinophil count >0.3 × | | | |
| 10³/μL | | | |
| Placebo | 12 | 2.09 (0.77, 4.55) | |
| Benralizumab 0.3 mg/kg | 7 | 1.21 (0.15, 4.38) | .505 |
| Benralizumab 1.0 mg/kg | 7 | 1.79 (0.37, 5.22) | .824 |
| Benralizumab total | 14 | 1.50 (0.49, 3.51) | .586 |

^{a}Blood samples for eosinophil counts were collected at screening and analyzed at the local lab.

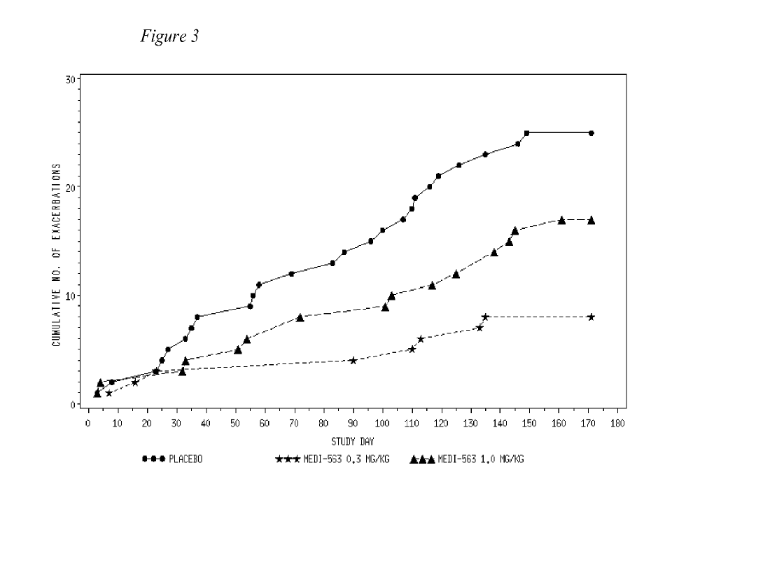

The number of subjects who experienced ≧1 asthma exacerbation resulting in hospitalization through week 12 was 7/36 (19.4%) vs. 8/72 (11.1%; P=0.25) for the placebo vs. combined treatment groups, respectively. The number of exacerbations resulting in hospitalization through week 12 was 14 from 36 evaluable subjects in the placebo group and 11 from 72 evaluable subjects in the combined treatment group (FIG. 3). Compared to placebo, the weighted adjudicated rate of exacerbations resulting in hospitalization (exacerbations/subject/year) in the combined treatment groups was reduced by 60% (Table 3). The weighted rate of exacerbations resulting in hospitalization was 1.62 (95% CI: 0.89-2.72) for placebo vs. 0.35 (95% CI: 0.07-1.02; P=0.016) for the 0.3 mg/kg group with a relative risk of 4.64 (95% CI: 1.30-25.19), 0.95 (95% CI: 0.41-1.87; P=0.225) for the 1.0 mg/kg group with a relative risk of 1.71 (95% CI: 0.67-4.71), and 0.65 (95% CI: 0.32-1.16; P=0.022) for the combined treatment groups with a relative risk of 2.51 (95% CI: 1.06-6.11).

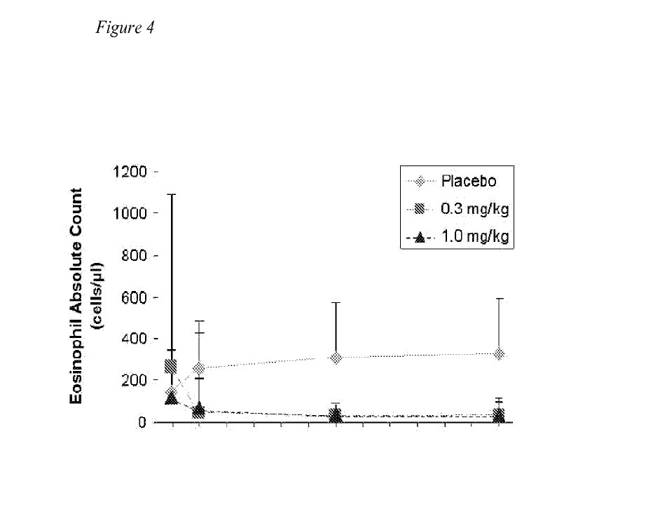

The effects of benralizumab with benralizumab on eosinophil counts is shown in FIG. 4 and Table 5. There is a marked reduction in eosinophil counts which was rapid and sustained to week 12 in both dose levels. Similar declines were produced by both dose levels in eosinophil-derived proteins: eosinophilic cationic protein (ECP) and eosinophil-derived neurotoxin (EDN). For the combined treatment groups, ECP declined from a baseline mean (SD) of 26.1 (30.3) μg/L to 8.6 (9.1) μg/L at day 84 and EDN declined from 25.67 (33.80) ng/mL to 4.79 (9.87) ng/mL.

TABLE 5
Eosinophils, Absolute (10\hat{ }3/μl)

| | PLACEBO | 0.3 MG/KG | 1.0 MG/KG | TOTAL |
|---|---|---|---|---|
| VISIT | (N = 38) | (N = 36) | (N = 36) | (N = 72) |
| DAY 0 | N 36 | 33 | 32 | 65 |
| MEAN | 0.146 | 0.267 | 0.122 | 0.195 |
| SD | 0.199 | 0.823 | 0.150 | 0.596 |
| MEDIAN | 0.070 | 0.040 | 0.045 | 0.040 |
| MIN-MAX | (0.00-0.88) | (0.00-4.53) | (0.00-0.50) | (0.00-4.53) |
| DAY 7 | N 33 | 33 | 29 | 62 |
| MEAN | 0.256 | 0.042 | 0.071 | 0.056 |
| SD | 0.226 | 0.166 | 0.354 | 0.269 |
| MEDIAN | 0.210 | 0.000 | 0.000 | 0.000 |
| MIN-MAX | (0.02-0.87) | (0.00-0.96) | (0.00-1.91) | (0.00-1.91) |
| DAY 42 | N 30 | 33 | 36 | 69 |
| MEAN | 0.306 | 0.012 | 0.017 | 0.014 |
| SD | 0.265 | 0.024 | 0.060 | 0.046 |
| MEDIAN | 0.200 | 0.000 | 0.000 | 0.000 |
| MIN-MAX | (0.06-1.23) | (0.00-0.12) | (0.00-0.36) | (0.00-0.36) |
| DAY 84 | N 34 | 34 | 31 | 65 |
| MEAN | 0.324 | 0.024 | 0.022 | 0.023 |
| SD | 0.264 | 0.062 | 0.084 | 0.073 |
| MEDIAN | 0.270 | 0.000 | 0.000 | 0.000 |
| MIN-MAX | (0.01-1.09) | (0.00-0.30) | (0.00-0.47) | (0.00-0.47) |

Benralizumab demonstrated no significant effects on pulmonary function, ACQ, or AQLQ when compared to placebo. Baseline values were established during the initial asthma exacerbation, and all of these measurements recovered similarly during the following 12 weeks in all 3 treatment groups (Table 6).

TABLE 6
Secondary outcome measures for the intent-to-treat population

| Secondary | | | | |
|---|---|---|---|---|
| Outcome | | Benralizumab | | |
| Measure | Placebo | 0.3 mg/kg | 1.0 mg/kg | Total |
| Mean (SD)/n | n = 38 | n = 36 | n = 36 | n = 72 |
| FEV₁, % predicted | | | | |
| Day 0 | 56.1 (15.8)/38 | 53.1 (17.8)/35 | 63.3 (22.8)/34 | 58.1 (20.9)/69 |
| Day 7 | 66.0 (14.3)/36 | 63.5 (16.4)/35 | 70.0 (23.2)/35 | 66.8 (20.2)/70 |
| Day 42 | 65.0 (17.6)/33 | 67.1 (17.5)/35 | 66.1 (21.6)/34 | 66.6 (19.5)/69 |
| Day 84 | 67.1 (17.9)/36 | 64.9 (17.3)/35 | 67.6 (21.9)/32 | 66.2 (19.5)/67 |
| FEV₁, L | | | | |
| Day 0 | 1.68 (0.52)/38 | 1.69 (0.70)/35 | 2.07 (0.75)/34 | 1.88 (0.74)/69 |
| Day 7 | 2.00 (0.63)/36 | 2.00 (0.66)/35 | 2.25 (0.72)/35 | 2.13 (0.70)/70 |
| Day 42 | 1.96 (0.71)/33 | 2.13 (0.71)/35 | 2.15 (0.72)/34 | 2.14 (0.71)/69 |
| Day 84 | 2.05 (0.76)/36 | 2.04 (0.71)/35 | 2.21 (0.79)/32 | 2.12 (0.75)/67 |
| PEF, L/min | | | | |
| Day 0 | 252.4 (95.6)/38 | 264.4 (99.8)/35 | 285.9 (109.8)/34 | 275.0 (104.6)/69 |
| Day 7 | 294.2 (82.6)/36 | 308.4 (96.1)/35 | 324.5 (114.0)/35 | 316.4 (105.0)/70 |
| Day 42 | 301.4 (102.4)/33 | 332.6 (91.4)/35 | 329.0 (120.4)/34 | 330.8 (105.9)/69 |
| Day 84 | 312.3 (109.2)/36 | 315.4 (97.1)/35 | 313.1 (127.2)/32 | 314.3 (111.6)/67 |
| ACQ | | | | |
| Day 0 | 3.64 (0.83)/38 | 3.72 (1.18)/36 | 3.26 (1.27)/36 | 3.49 (1.24)/72 |
| Day 7 | 1.89 (1.16)/36 | 1.89 (1.36)/36 | 1.52 (0.96)/35 | 1.71 (1.18)/71 |
| Day 42 | 1.70 (1.23)/33 | 1.76 (1.25)/36 | 1.50 (1.13)/36 | 1.63 (1.19)/72 |
| Day 84 | 1.59 (1.23)/36 | 1.75 (1.33)/35 | 1.70 (1.12)/32 | 1.73 (1.23)/67 |
| AQLQ | | | | |
| Day 0 | 3.10 (0.90)/38 | 3.18 (0.95)/36 | 3.35 (1.11)/36 | 3.27 (1.03)/72 |
| Day 42 | 4.74 (1.65)/33 | 4.76 (1.46)/36 | 5.16 (1.44)/36 | 4.96 (1.45)/72 |
| Day 84 | 4.91 (1.71)/35 | 5.01 (1.44)/35 | 5.11 (1.35)/32 | 5.06 (1.38)/67 |

Abbreviations:
ACQ, Asthma Control Questionnaire;
AQLQ, Asthma Quality of Life Questionnaire;
FEV₁, forced expiratory volume in 1 second;
PEF, peak expiratory force

Healthcare resource utilization is shown in Table 7. At Week 24, patient visits to the ED were significantly lower (p=0.02) for subjects treated with benralizumab (weighted rate of 4.32) compared to placebo (2.95).

TABLE 7
Healthcare resource utilization^{a}

| | | Benralizumab | | | |
|---|---|---|---|---|---|
| Rate^{b} (No. | Placebo | 0.3 mg/kg | 1.0 mg/kg | Total | |
| of Events) | n = 36 | n = 36 | n = 36 | n = 72 | P Value^{c} |
| ICU admits | 0.12 (1) | 0 | 0 | 0 | 1.00 |
| 12 wk | | | | | |
| ICU admits | 0.27 (4) | 0.07 (1) | 0.07 (1) | 0.07 (2) | 0.12 |
| 24 wk | | | | | |
| ICU days | 14 | 2 | 8 | 10 | 0.27^{d} |
| 24 wk | | | | | |
| Hosp admits | 1.51 (13) | 0.82 (7) | 0.95 (8) | 0.88 (15) | 0.16 |
| 12 wk | | | | | |
| Hosp admits | 1.66 (25) | 0.88 (13) | 1.26 (18) | 1.06 (31) | 0.10 |
| 24 wk | | | | | |
| Hosp days | 73 | 38 | 82 | 120 | 0.73^{d} |
| 24 wk | | | | | |
| ED visits | 4.40 (38) | 3.73 (32) | 2.96 (25) | 3.35 (57) | 0.19 |
| 12 wk | | | | | |
| ED visits | 4.32 (65) | 2.83 (42) | 3.07 (44) | 2.95 (86) | 0.02 |
| 24 wk | | | | | |

Abbreviations:
ICU, intensive care unit;
ED, emergency department.
^{a}Values are for the evaluable population.
^{b}Rate for each group = total events/total duration of person-year follow-up.
^{c}Placebo vs. total benralizumab; statistics performed using Pairwise Poisson regression unless otherwise indicated
^{d}Pairwise t-test

### (C) Safety

Adverse events (AE) occurring in ≧5% of subjects treated with benralizumab were asthma, headache, dizziness, cough, pyrexia, bronchitis, anxiety, muscle spasms, and hyperhidrosis. All AEs were mild to moderate in severity and self-limiting (Table 8). The numbers of severe adverse events were similar in number among groups. The number of subjects who experienced serious AEs considered related to benralizumab was: pyrexia (n=2), tachycardia (n=1), and anxiety (n=1). Six subjects in the treated group had anti-drug antibodies at Week 12 without any clinical sequelae.

TABLE 8
Adverse events in ≧5% of subjects by treatment group

| | | Benralizumab | | |
|---|---|---|---|---|
| Adverse event | Placebo | 0.3 mg/kg | 1.0 mg/kg | Total |
| No. Subjects (%) | n = 38 | n = 36 | n = 36 | n = 72 |
| Asthma | 22 (57.9) | 15 (41.7) | 19 (52.8) | 34 (47.2) |
| Headache | 4 (10.5) | 5 (13.9) | 9 (25.0) | 14 (19.4) |
| Dizziness | 1 (2.6) | 5 (13.9) | 3 (8.3) | 8 (11.1) |
| Cough | 3 (7.9) | 1 (2.8) | 6 (16.7) | 7 (9.7) |
| Pyrexia | 1 (2.6) | 2 (5.6) | 3 (8.3) | 5 (6.9) |
| Bronchitis | 3 (7.9) | 2 (5.6) | 2 (5.6) | 4 (5.6) |
| Anxiety | 1 (2.6) | 1 (2.8) | 3 (8.3) | 4 (5.6) |
| Muscle spasms | 1 (2.6) | 2 (5.6) | 2 (5.6) | 4 (5.6) |
| Hyperhidrosis | 0 (0.0) | 2 (5.6) | 2 (5.6) | 4 (5.6) |
| Wheezing | 2 (5.3) | 2 (5.6) | 1 (2.8) | 3 (4.2) |
| Nausea | 1 (2.6) | 0 (0.0) | 3 (8.3) | 3 (4.2) |
| Blood creatinine | 0 (0.0) | 0 (0.0) | 3 (8.3) | 3 (4.2) |
| increased | | | | |
| Dyspepsia | 0 (0.0) | 3 (8.3) | 0 (0.0) | 3 (4.2) |
| Procedural pain | 0 (0.0) | 2 (5.6) | 1 (2.8) | 3 (4.2) |
| Sinusitis | 0 (0.0) | 2 (5.6) | 1 (2.8) | 3 (4.2) |
| Back pain | 3 (7.9) | 0 (0.0) | 2 (5.6) | 2 (2.8) |
| Nasal congestion | 3 (7.9) | 2 (5.6) | 0 (0.0) | 2 (2.8) |
| Nasopharyngitis | 2 (5.3) | 0 (0.0) | 2 (5.6) | 2 (2.8) |
| Pneumonia | 2 (5.3) | 1 (2.8) | 1 (2.8) | 2 (2.8) |
| Diarrhea | 1 (2.6) | 0 (0.0) | 2 (5.6) | 2 (2.8) |
| Fall | 1 (2.6) | 0 (0.0) | 2 (5.6) | 2 (2.8) |
| Fatigue | 1 (2.6) | 0 (0.0) | 2 (5.6) | 2 (2.8) |
| Insomnia | 1 (2.6) | 0 (0.0) | 2 (5.6) | 2 (2.8) |
| Oropharyngeal pain | 1 (2.6) | 0 (0.0) | 2 (5.6) | 2 (2.8) |
| Constipation | 0 (0.0) | 0 (0.0) | 2 (5.6) | 2 (2.8) |
| Night sweats | 0 (0.0) | 2 (5.6) | 0 (0.0) | 2 (2.8) |
| Vomiting | 0 (0.0) | 0 (0.0) | 2 (5.6) | 2 (2.8) |
| Upper respiratory | 3 (7.9) | 1 (2.8) | 0 (0.0) | 1 (1.4) |
| tract infection | | | | |
| Arthralgia | 2 (5.3) | 1 (2.8) | 0 (0.0) | 1 (1.4) |
| Myalgia | 2 (5.3) | 0 (0.0) | 1 (2.8) | 1 (1.4) |
| Dehydration | 2 (5.3) | 0 (0.0) | 0 (0.0) | 0 (0.0) |
| Hypokalemia | 2 (5.3) | 0 (0.0) | 0 (0.0) | 0 (0.0) |
| Joint sprain | 2 (5.3) | 0 (0.0) | 0 (0.0) | 0 (0.0) |

### (d) Discussion

This study demonstrates that one dose of benralizumab significantly reduced the blood eosinophil count, the number and severity of subsequent exacerbations, and healthcare utilization in subjects who presented to the ED with a severe asthma exacerbation. It did not, however, impact the proportion of subjects who experienced ≧1 subsequent exacerbation or important indicators such as pulmonary function and self-reported quality of life. However, the proportion of subjects who experienced ≧2 exacerbations was significantly lower compared to placebo (P<0.05).

Patients with acute asthma who are discharged from a hospital or ED setting are at risk for relapse and future exacerbations. Subjects in this study had severe, poorly controlled asthma as evidenced by a past history of multiple ED visits, hospital and ICU admissions. Comparable patient populations have proven to be among the most difficult to achieve acceptable asthma control (Moore W C, Peters S P. J Allergy Clin Immunol 117:487-494 (2006)). Best evidence suggests systemic (Rowe B H et al. Acad Emerg Med 15:708-717 (2008)) and inhaled (Rowe B H et al. JAMA 281:2119-2126 (1999)) corticosteroids are required to regain and maintain control after discharge. Previous studies have also demonstrated that an outpatient monitoring strategy focused on reducing eosinophils in the sputum reduces the number of subsequent asthma exacerbations (Bellido-Casado J, et al. Arch Bronconeumol 46(11): 587-93 (2010); Green R, et al. Lancet 360:1715-21 (2002)). This study extends this line of reasoning to unstable patients who present to the ED with an asthma exacerbation that is severe and poorly responsive to standard therapy with bronchodilators and systemic corticosteroids. Treatment of these subjects with a single dose of benralizumab following a severe asthma exacerbation resulted in a significant reduction in the number of exacerbations requiring an urgent care visit over the subsequent 12 weeks. Of note, the number of hospital admissions at 12 weeks due to asthma was reduced by 60% in the combined treatment groups as compared to standard therapeutic regimens—particularly in subjects with multiple exacerbations. This result is comparable to that previously reported in a similar patient population (Haldar P, et al. N Engl J Med 360:973-84 (2009)).

While not wishing to be bound by theory, the results also suggest that there is a persistent effect of a single dose of benralizumab on exacerbations beyond 12 weeks. However, these data are limited because only 80% of 108 evaluable subjects were followed from week 12 through 24 weeks by telephone contact to inquire about asthma exacerbations and related adverse events.

There are two potential explanations for the effectiveness of benralizumab after a severe asthma exacerbation. Studies of near fatal asthma (Restrepo R and Peters J. Curr Opin Pulm Med 14: 13-23 (2008)) and sputum cell counts in patients presenting to the ED with an asthma exacerbation (Bellido-Casado J, et al. Arch Bronconeumol 46(11): 587-93 (2010)) suggest that the majority of these patients have an increase in airway eosinophils. A course of systemic corticosteroids given in the ED or hospital on discharge is effective in reducing asthma exacerbations over the subsequent 21 days (Rowe B H et al. Cochrane Database Syst Rev 3:CD000195 (2007)). Benralizumab achieves greater reductions in eosinophils and basophils to levels that cannot be achieved by systemic corticosteroids both in the blood and in the airways and for a sustained treatment effect over a long period of time (Busse W, et al. JACI 125: 1237-43 (2010); Kolbeck R, et al. JACI 125:1344-53 (2010)).

While again not wishing to be bound by theory, another possibility is that patients who repeatedly present to the ED with poorly controlled asthma have incomplete adherence to post-ED controller medications. Patients in the benralizumab treated group may have benefited from the prolonged depletion of eosinophils independent of their compliance with standard therapy. While compliance with standard therapy was measured by self-report, this measure is unreliable and poses a limitation of this study.

The current study did not select subjects based on elevated blood or sputum eosinophils; instead, an “all-comers” (with exacerbations likely to be eosinophilic) strategy was used. Subjects were stratified by baseline eosinophil count of greater or less than 450 eosinophils/mm³ to ensure an even distribution of patients at increased risk of early relapse (Janson C and Herala M. Resp Med 86(2):101-104 (1992)). Although subjects with increased sputum eosinophils were not specifically targeted, patients who present to the ED with an asthma exacerbation have demonstrated elevated sputum and blood eosinophils (Bellido-Casado J, et al. Arch Bronconeumol 46:587-93 (2010)). In addition, a poor response to bronchodilators has been associated with eosinophilic airway inflammation and a favorable response to anti-eosinophilic therapy (Pavord I D, et al. Thorax 65:370 (2010)). These characteristics may explain the response to benralizumab in this particular patient population.

The best method for determining an eosinophilic exacerbation in the acute setting is not well established. Sputum analysis is not practical in the ED, and blood eosinophil counts are at best poor predictors of increased airway eosinophils (Pizzichini E, et al. J Allergy Clin Immunol. 99:539-44 (1997)). Although one would expect benralizumab to demonstrate a greater clinical effect in subjects who experience predominantly eosinophilic exacerbations, subjects in this study demonstrated a clinical response to benralizumab regardless of whether they had a high or low baseline eosinophil count. However, a significant number of subjects had been exposed to systemic corticosteroids prior to measurement of baseline eosinophil counts that were low or not measurable, confounding this analysis. These data suggest that blood eosinophil measurements do not appear to be a particularly useful predictor of clinical response in this setting.

An unusual aspect of this study was the dose-response relationship: the 0.3 mg/kg treatment group fared better than the 1.0 mg/kg treatment group. While not wishing to be bound by theory, the two doses had essentially the same effect on PB eosinophils suggesting that these dose levels were comparable and the observed differences may have been due to underlying baseline differences in the study groups. For example, the 1.0 mg/kg group had more tobacco and marijuana smokers, which has been reported to result in more characteristics of chronic obstructive pulmonary disease (Tetrault J M et al. Arch Intern Med 167:221-228 (2007)) and a greater number of asthma exacerbations (Gaeta T J, et al. Acad Emerg Med 3:1170-1172 (1996)). Yet, the 1.0 mg/kg group still fared better than the placebo group with regards to asthma exacerbations and hospitalizations.

While not being bound by theory, a patient's response to beta-agonist therapy and corticosteroids may predict clinical response to an anti-eosinophil treatment. When treated with mepolizumab, an anti-IL-5 monoclonal antibody, subjects with the least improvement in FEV1 after beta-agonist administration and best response to a course of oral corticosteroids had the greatest improvement in exacerbation rates. Conversely, those patients with a good response to bronchodilators and a poor response to corticosteroids had the least improvement in exacerbation rates (Pavord I D, et al. Thorax 65:370 (2010)). In the current study, baseline percent-predicted FEV1 can be used as a proxy for bronchodilator response because the majority of these measurements were taken after receiving bronchodilator treatment in the ED. The 0.3 mg/kg treatment group had the lowest baseline FEV₁, indicating a poor response to bronchodilators, and the best response at Day 7 after a week of corticosteroid treatment. In comparison, the 1.0 mg/kg treatment group had the highest baseline FEV₁ and least improvement after a week of corticosteroids, which may suggest that this group was generally less likely to respond to treatment with an anti-eosinophil medication (Table 9).

TABLE 9
Post-hoc Summary of Asthma Controller Use, Evaluable Population

| | | | Benralizumab, 0.3 mg/kg | | Benralizumab, 1.0 mg/kg | |
|---|---|---|---|---|---|---|
| | Placebo (n = 36) | | (n = 36) | | (n = 36) | |
| | Number of | Number of | Number of | Number of | Number of | Number of |
| | subjects | subjects | subjects | subjects | subjects | subjects |
| | with | with | with | with | with | with |
| | controller | controller | controller | controller | controller | controller |
| | use at | use on | use at | use on | use at | use on |
| | entry^{a} | study^{b} | entry^{a} | study^{b} | entry^{a} | study^{b} |
| ICS | 8 (22.2%) | 12 (33.3%) | 9 (25.0%) | 3 (8.3%) | 6 (16.7%) | 9 (25.0%) |
| ICS/LABA | 22 (61.1%) | 11 (30.6%) | 20 (55.6%) | 13 (36.1%) | 19 (52.8%) | 10 (27.8%) |
| LTRI | 12 (33.3%) | 5 (13.9%) | 9 (25.0%) | 9 (25.0%) | 8 (22.2%) | 3 (8.3%) |
| LABA | 4 (11.1%) | 1 (2.8%) | 5 (13.9%) | 2 (5.6%) | 2 (5.6%) | 2 (5.6%) |
| LAMA | 0 (0.0%) | 0 (0.0%) | 2 (5.6%) | 2 (5.6%) | 1 (2.8%) | 3 (8.3%) |

Abbreviations:
ICS inhaled corticosteroid;
LABA, long-acting beta agonist;
LTRI, leukotriene modifier;
LAMA, long-acting muscarinic agonist.
^{a}The controller use at entry includes an asthma medication with onset date of study day-7 or earlier.
^{b}The controller use on study includes an asthma medication with onset date of study day 0 or later.

Despite the reduction in the exacerbation rate, benralizumab had little impact on other dimensions of asthma care such as pulmonary function, asthma control, and asthma quality of life. No significant safety issues with benralizumab were identified during this or a prior study (Busse W, et al. JACI 125: 1237-1244 e2 (2010)).

Benralizumab, by merit of the sustained reduction in eosinophils, can be used to mitigate the risk for subsequent severe asthma exacerbations in this population. The presence of an effective therapy in this setting can provide an impetus to identify these patients and to improve medical care in this underserved patient population.

In conclusion, treatment with a single dose of benralizumab resulted in a long lasting reduction of eosinophils and in the number and severity of exacerbations in subjects with asthma who presented to the ED with a severe exacerbation.

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Those skilled in the art will recognize, or be able to ascertain using no more than routine experimentation, many equivalents to the specific aspects of the invention described herein. Such equivalents are intended to be encompassed by the following claims.

Although the foregoing invention has been described in some detail by way of illustration and example for purposes of clarity of understanding, it will be obvious that certain changes and modifications can be practiced within the scope of the appended claims.

What is claimed is:

1. A method of reducing the number and severity of acute exacerbations of asthma in an asthma patient, comprising administering to a patient with a history of acute exacerbations of asthma an effective amount of an anti-interleukin-5 receptor (IL-5R) antibody or antigen-binding fragment thereof.

2. The method of claim 1, wherein the patient's severe acute exacerbations are characterized by one or more symptoms selected from the group consisting of: (a) wheezing; (b) dyspnea; (c) a forced expiratory volume in one second (FEV₁) no more than 60%, 70%, or 80% of predicted value; (d) a peak expiratory flow (PEF) of no more than 60%, 70%, or 80% of predicted value; (e) coughing; and (f) two or more of the symptoms.

3. The method of claim 1 or claim 2, wherein the patient's exacerbations are refractory to bronchodilator treatments.

4. The method of any one of claims 1 to 3, which reduces the number of acute exacerbations over a 12-week period following administration of the antibody or antigen-binding fragment thereof, as compared to the number of exacerbations expected according to the patient's history.

5. The method of any one of claims 1 to 4, which reduces the number of acute exacerbations over a 24-week period following administration of the antibody or antigen-binding fragment thereof, as compared to the number of exacerbations expected according to the patient's history.

6. The method of claim 4, wherein the number of recurring exacerbations is reduced by at least 40% over the 12-week period.

7. The method of any one of claims 1 to 6, which reduces the severity of one or more acute exacerbations, as compared to the severity of exacerbations expected according to the patient's history.

8. The method of claim 7, wherein the number of exacerbations requiring an Emergency Department visit is reduced by at least 50% over a 12-week period.

9. The method of claim 7 or claim 8, wherein the number of exacerbations requiring hospitalization is reduced by at least 40% over a 12-week period.

10. The method of claim 9, wherein the length of any required hospitalizations is reduced.

11. The method of claim 9 or claim 10, wherein the number of hospitalizations requiring ICU admission is reduced.

12. The method of any one of claims 1 to 11, wherein the antibody or antigen binding fragment thereof is a monoclonal antibody.

13. The method of claim 12, wherein the antibody or antigen binding fragment thereof is a chimeric antibody.

14. The method of claim 12, wherein the antibody or antigen binding fragment thereof is a humanized antibody.

15. The method of claim 12, wherein the antibody or antigen binding fragment thereof is a human antibody.

16. The method of any of claims 1 to 15, wherein the antibody or antigen-binding fragment thereof specifically binds to the IL-5R α chain.

17. The method of any one of claims 1 to 16, wherein the antibody or antigen-binding fragment thereof further comprises a constant region.

18. The method of claim 17, where the constant region comprises an immunoglobulin Fc region.

19. The method of claim 18, wherein the immunoglobulin Fc region is altered in a manner that increases effector function.

20. The method of claim 19, wherein the immunoglobulin Fc region comprises reduced levels of fucose.

21. The method of claim 20, wherein the immunoglobulin Fc region comprises no fucose.

22. The method of any one of claims 19 to 21, wherein the immunoglobulin Fc region comprises amino acid substitutions that yield increased effector function.

23. The method of claim 22, wherein the amino acid substitutions comprise the inclusion of the following amino acid sequences in the Fc region: 332E. 239D and 330L. as numbered by the EU index as set forth in Kabat.

24. The method of any one of claims 1 to 23, wherein the antibody or antigen-binding fragment thereof binds to the same IL-5Rα epitope as benralizumab.

25. The method of claim 24, wherein the antibody or antigen-binding fragment thereof is benralizumab or an antigen-binding fragment thereof.

26. The method of any one of claims 1 to 25, wherein the anti-IL-5R antibody or antigen-binding fragment thereof is administered as a single dose.

27. The method of any one of claims 1 to 26, wherein the anti-IL5R antibody or antigen-binding fragment thereof is administered as two or more doses.

28. The method of claim 27, wherein the two or more doses are spaced at least five (5) weeks apart.

29. The method of claim 30, wherein the two or more doses are spaced at least twelve (12) weeks apart.

30. The method of any one of claims 26 to 29, wherein the single dose or first dose is administered to the patient within 7 days of an acute exacerbation of asthma.

31. The method of any one of claims 1 to 30, wherein the anti-IL5R antibody or antigen-binding fragment thereof is administered at a dose of between about 0.1 mg/kg and 2 mg/kg per dose.

32. The method of claim 31, wherein the dose is 0.3 mg/kg.

33. The method of claim 31, wherein the dose is 1 mg/kg.

34. The method of any one of claims 1 to 33, wherein the anti-IL5R antibody or antigen-binding fragment thereof is administered parenterally.

35. The method of claim 34, wherein the anti-IL5R antibody or antigen-binding fragment thereof is administered intravenously.

36. The method of any one of claims 1 to 35, wherein the antibody or antigen-binding fragment thereof is administered in addition to corticosteroid therapy.

37. The method of any one of claims 1 to 36, wherein the patient's acute exacerbations are severe.

38. The method of any one of claims 1 to 37, wherein a course of systemic corticosterioids given in the hospital on discharge is not completely effective at reducing eosinophil count in the patient.

39. The method of any one of claims 1 to 38, wherein the patient is not fully compliant with standard post-hospital therapy.

40. The method of any one of claims 1 to 39, and wherein the anti-IL5R antibody or antigen-binding fragment thereof depletes eosinophil count independent of compliance with standard therapy.

41. The method of any one of claims 1 to 40, wherein the patient presents with a characteristic selected from the group consisting of an elevated circulating eosinophil count, an elevated eosinophil count in induced sputum, an elevated eosinophil cationic protein level, an elevated eosinophil-derived neurotoxin level and a combination of the recited characteristics.

42. The method of any one of claims 1 to 40, wherein the patient presents with a characteristic selected from the group consisting of a normal circulating eosinophil count, a normal eosinophil count in induced sputum, a normal eosinophil cationic protein level, a normal eosinophil-derived neurotoxin level and a combination of the recited characteristics.
